(19) United States
(12) Patent Application Publication
(10) Pub. No.: US 2013/0197229 A1
(43) Pub. Date: Aug. 1, 2013

(54) METHOD OF MAKING AZAINDAZOLE DERIVATIVES

(71) Applicants: Christopher Matthews, San Diego, CA (US); Colin O'Bryan, Cardiff-by-the-Sea, CA (US); David Paul Provencal, san Diego, CA (US)

(72) Inventors: Christopher Matthews, San Diego, CA (US); Colin O'Bryan, Cardiff-by-the-Sea, CA (US); David Paul Provencal, san Diego, CA (US)

(21) Appl. No.: 13/878,412

(22) PCT Filed: Oct. 13, 2011
(86) PCT No.: PCT/US11/56208

§ 371 Date: Apr. 8, 2013

Related U.S. Application Data
(60) Provisional application No. 61/392,904, filed on Oct. 13, 2010.

(51) Int. Cl. C07D 471/04; C07D 309/06

(52) U.S. Cl. CPC C07D 471/04; C07D 309/06
USPC 546/119; 549/427

(57) ABSTRACT

Disclosed are methods, reagents, and intermediates useful for making azaindazole derivatives, which may be used to modulate Glucokinase. The disclosed methods and materials are generally useful for making halo-esters and sulfonyl-substituted compounds.

### FIELD OF THE INVENTION

The present invention relates to methods, reagents, and intermediates useful for making aliphatic or aromatic sulfonyl-substituted azaindazole compounds, which are activators of Glucokinase.

### BACKGROUND OF THE INVENTION

Glucokinase (GK, Hexokinase IV) is one of four hexokinases that are found in mammals (Colowick, S. P., in The Enzymes, Vol. 9 (P. Boyer, ed.) Academic Press, New York, N.Y., pages 1-48, 1973). Compounds that activate GK are expected to be useful in the treatment of hyperglycemia, which is characteristic of type II diabetes.

Activators of GK are known in the art. See, for example, WO 2004/072031 A2 and WO 2004/072066 A1 (OSI); WO 2007/051847 A1 and WO 06/016194 A1 (Prosidion); WO 03/055482 A1, WO 2004/002481 A1, WO 2005/049019 A1, and WO 2008/084043 A1 (Novo Nordisk); WO 2007/122482 A1 and US 2008/0280875 A1 (Pfizer); WO 2007/041365 A2 (Novartis); and WO 2008/005964 A2 (BMS).

International patent application WO 2009/140624 A2 (the “'624 Application”) describes a number of aliphatic and aromatic sulfonyl-substituted azaindazole compounds, which are potent activators of GK. The '624 Application describes useful methods for preparing the azaindazole derivatives at laboratory scale. However, some of the methods may be less suitable for pilot plant or commercial scale because they employ expensive starting materials (e.g., sodium cyclopropyl sulfinate), high temperatures (e.g., >120° C.), and chromatographic separations, among other things.

### SUMMARY OF THE INVENTION

The present invention provides methods and materials for preparing aliphatic or aromatic sulfonyl-substituted azaindazole compounds and useful reaction intermediates.

One aspect of the invention provides a method of making compounds of formula 1,

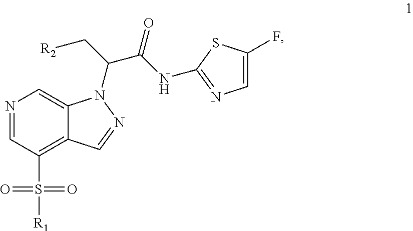

or a pharmaceutically acceptable salt thereof, the method comprising:

reacting a compound of formula A3

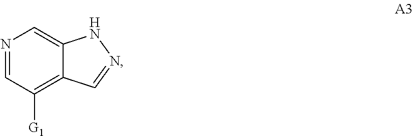

with a compound of formula A4,

(R₁—S(O)₂)₂Zn, A4

to give a compound of formula A5,

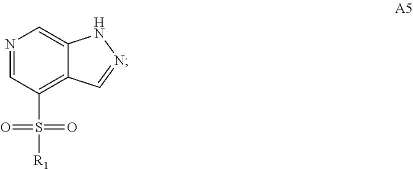

reacting the compound of formula A5 with a compound of formula A6,

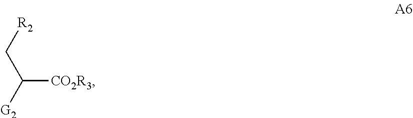

to give, following hydrolysis, a compound of formula A7,

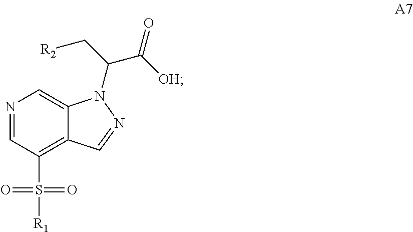

reacting the compound of formula A7 with a compound of formula A9,

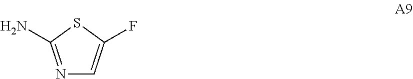

or a salt thereof, to give the compound of formula 1; and

optionally converting the compound of formula 1 to a pharmaceutically acceptable salt;

wherein

G₁ and G₂ are each independently halo;

R₁ is selected from the group consisting of C₁₋₆ alkyl, C₃₋₈ cycloalkyl-C₁₋₆ alkyl, C₃₋₆ heterocycloalkyl-C₁₋₅ alkyl, C₆₋₁₄ aryl-C₁₋₆ alkyl, C₁₋₁₀ heteroaryl-C₁₋₆ alkyl, C₃₋₈ cycloalkyl, C₃₋₆ heterocycloalkyl, C₆₋₁₂ aryl, and C₁₋₁₀ heteroaryl, each optionally substituted;

R₂ is selected from the group consisting of hydrogen, halo, cyano, thio, hydroxy, C₁₋₅ carbonyloxy, C₁₋₄ alkoxy, C₆₋₁₄ aryloxy, C₁₋₁₀ heteroaryloxy, C₁₋₅ oxycarbonyl, C₁₋₉ amide, C₁₋₇ amido, C₀₋₈ alkylamino, C₁₋₆ sulfonylamido, imino, C₁₋₈ sulfonyl, C₁₋₆ alkyl, C₃₋₈ cycloalkyl-C₁₋₆ alkyl, C₃₋₆ heterocycloalkyl-C₁₋₆ alkyl, C₆₋₁₄ aryl-C₁₋₆ alkyl, C₁₋₁₀ heteroaryl-C₁₋₅ alkyl, C₃₋₈ cycloalkyl, C₃₋₆ heterocycloalkyl, C₆₋₁₄ aryl, and C₁₋₁₀ heteroaryl, each optionally substituted; and

R₃ is selected from the group consisting of (C₁₋₆)alkyl, (C₃₋₈)cycloalkyl, (C₃₋₆)heterocycloalkyl, (C₆₋₁₄)aryl, (C₁₋₁₀)heteroaryl, (C₃₋₈)cycloalkyl(C₁₋₆)alkyl, (C₃₋₆)heterocycloalkyl(C₁₋₆)alkyl, (C₆₋₁₄)aryl(C₁₋₆)alkyl, and (C₁₋₁₀)heteroaryl(C₁₋₆)alkyl, each optionally substituted.

Another aspect of the invention provides a method of making compounds of formula C2,

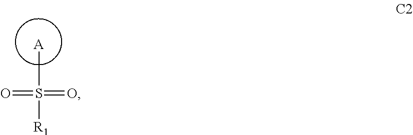

the method comprising:

reacting a compound of formula C1,

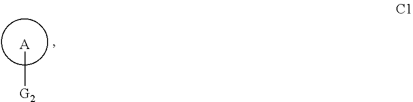

with a compound formula A4,

(R₁—S(O)₂)₂Zn; A4

wherein

A is selected from the group consisting of C₃₋₈ cycloalkyl, C₃₋₆ heterocycloalkyl, C₆₋₁₄ aryl, and C₁₋₁₀ heteroaryl, each optionally substituted; and

G₂ and R₁ are as defined above.

A further aspect of the invention provides a method of making compounds of formula A5,

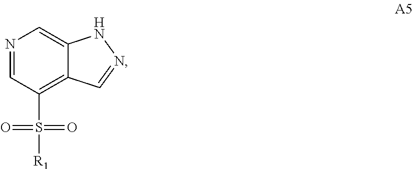

the method comprising:

reacting a compound of formula A3,

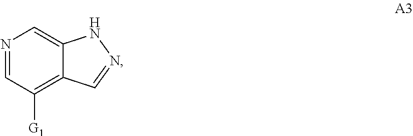

with a compound of formula A4,

(R₁—S(O)₂)₂Zn; A4

wherein G₁ and R₁ are as defined above.

An additional aspect of the invention provides a method of making compounds of formula A6,

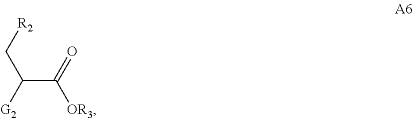

the method comprising:

halogenating a compound of formula B6,

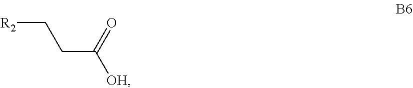

to give a compound of formula B7,

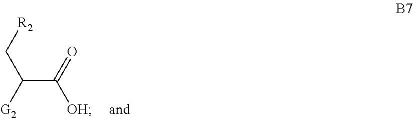

reacting the compound of formula B7 with R₃—OH, wherein G₂, R₂, and R₃ are as defined above.

### DEFINITIONS

Unless otherwise stated, the following terms used in the specification and claims shall have the following meanings.

It is noted that, as used in the specification and the appended claims, the singular forms “a,” “an” and “the” include plural referents unless the context clearly dictates otherwise. Further, definitions of standard chemistry terms may be found in reference works, including Carey and Sundberg, Advanced Organic Chemistry, 4th ed, vols. A (2000) and B (2001). Also, unless otherwise indicated, conventional methods of mass spectroscopy, NMR, HPLC, protein chemistry, biochemistry, recombinant DNA techniques and pharmacology, within the skill of the art are employed.

The term “C₁₋₆ alkyl” refers to a straight or branched alkyl chain having from one to six carbon atoms.

The term “optionally substituted C₁₋₆ alkyl” refers to a C₁₋₆ alkyl optionally having from 1 to 7 substituents independently selected from the group consisting of C₀₋₈ alkylamino, optionally substituted C₁₋₄ alkoxy, C₁₋₄ thioalkoxy, C₁₋₉ amide, C₁₋₅ oxycarbonyl, C₁₋₈ sulfonyl, cyano, optionally substituted C₃₋₈ cycloalkyl, halo, hydroxy, oxo, optionally substituted C₁₋₁₀ heteroaryl, optionally substituted C₃₋₆ heterocycloalkyl, optionally substituted C₁₋₁₀ heteroaryl, and optionally substituted phenyl.

More particularly “optionally substituted C₁₋₆ alkyl” refers to a C₁₋₆ alkyl optionally having from 1 to 7 substituents independently selected from the group consisting of C₁₋₄ alkoxy, C₁₋₉ amide, C₀₋₈ alkylamino, C₁₋₅ oxycarbonyl, cyano, C₃₋₈ cycloalkyl, halo, hydroxy, C₃₋₆ heterocycloalkyl optionally substituted on any ring nitrogen by C₁₋₄ alkyl, C₁₋₁₀ heteroaryl, and optionally substituted phenyl.

The term “C₁₋₈ sulfonyl” refers to a sulfonyl linked to a C₁₋₆ alkyl group, C₃₋₈ cycloalkyl, or an optionally substituted phenyl.

The term “C₁₋₄ alkoxy” refers to a C₁₋₄ alkyl attached through an oxygen atom.

The term “optionally substituted C₁₋₄ alkoxy” refers to a C₁₋₄ alkoxy optionally having from 1 to 6 substituents independently selected from the group consisting of C₁₋₄ alkoxy, C₁₋₉ amide, C₁₋₅ oxycarbonyl, cyano, optionally substituted C₃₋₈ cycloalkyl, halo, hydroxy, optionally substituted C₁₋₁₀ heteroaryl, and optionally substituted phenyl. While it is understood that where the optional substituent is C₁₋₄ alkoxy, cyano, halo, or hydroxy then the substituent is generally not alpha to the alkoxy attachment point, the term “optionally substituted C₁₋₄ alkoxy” includes stable moieties and specifically includes trifluoromethoxy, difluoromethoxy, and fluoromethoxy.

More particularly “optionally substituted C₁₋₄ alkoxy” refers to a C₁₋₄ alkoxy optionally having from 1 to 6 substituents independently selected from the group consisting of C₁₋₄ alkoxy, cyano, C₃₋₈ cycloalkyl, halo, hydroxy, and phenyl.

The term “C₁₋₉ amide” refers to an amide having two groups independently selected from the group consisting of hydrogen, C₁₋₄ alkyl, and optionally substituted phenyl. Examples include —CONH₂, —CONHCH₃, and —CON(CH₃)₂.

The term “C₁₋₂ amido” refers to a —NHC(O)R group in which R is selected from the group consisting of hydrogen, C₁₋₆ alkyl, and optionally substituted phenyl.

The term “C₁₋₅ carbamoyl” refers to an O— or N-linked carbamate having a terminal C₁₋₄ alkyl substituent.

The term “C₁₋₅ ureido” refers to a urea optionally having a C₁₋₄ alkyl substituent.

The term “C₀₋₈ alkylamino” refers to an amino optionally having one or two C₁₋₄ alkyl substituents.

The term “C₆₋₁₄ aryl” refers to a monocyclic or polycyclic unsaturated, conjugated hydrocarbon having aromatic character and having six to fourteen carbon atoms, and includes phenyl, biphenyl, indenyl, cyclopentyldienyl, fluorenyl, and naphthyl.

More particularly “C₆₋₁₄ aryl” refers to phenyl.

The term “optionally substituted C₆₋₁₄ aryl” refers to a C₆₋₁₄ aryl optionally having 1 to 5 substituents independently selected from the group consisting of C₀₋₈ alkylamino, C₁₋₇ amido, C₁₋₉ amide, C₁₋₅ carbamoyl, C₁₋₆ sulfonylamido, C₀₋₆ sulfonylamino, C₁₋₅ ureido, C₁₋₄ alkyl, C₁₋₄ alkoxy, cyano, halo, hydroxy, C₁₋₅ oxycarbonyl, trifluoromethyl, trifluoromethoxy, and C₁₋₈ sulfonyl.

More particularly “optionally substituted C₆₋₁₄ aryl” refers to a C₆₋₁₄ aryl optionally having 1 to 5 substituents independently selected from the group consisting of C₁₋₄ alkyl, C₁₋₄ alkoxy, cyano, halo, C₁₋₅ oxycarbonyl, trifluoromethyl, and trifluoromethoxy.

The term “C₆₋₁₄ aryloxy” refers to a C₆₋₁₄ aryl attached through an oxygen atom.

The term “optionally substituted C₆₋₁₄ aryloxy” refers to a C₆₋₁₄ aryloxy optionally having 1 to 5 substituents independently selected from the group consisting of C₀₋₈ alkylamino, C₁₋₄ alkyl, C₁₋₄ alkoxy, cyano, halo, hydroxy, nitro, C₁₋₈ sulfonyl, and trifluoromethyl.

The term “C₁₋₅ oxycarbonyl” refers to an oxycarbonyl group —CO₂H and C₁₋₄ alkyl ester thereof.

The term “C₁₋₅ carbonyloxy” refers to a carbonyloxy group —OC(O)R, where R is C₁₋₄ alkyl.

The term “C₃₋₈ cycloalkyl” refers to an alkyl ring having from three to eight carbon atoms, and includes cyclopropyl, cyclobutyl, cyclopentyl, cyclohexyl, and the like.

The term “optionally substituted C₃₋₈ cycloalkyl” refers to a C₃₋₈ cycloalkyl optionally having from 1 to 6 substituents independently selected from the group consisting of optionally substituted C₁₋₄ alkyl, optionally substituted C₁₋₄ alkoxy, C₁₋₆ amide, C₁₋₇ amido, C₀₋₈ alkylamino, C₁₋₅ oxycarbonyl, cyano, C₃₋₈ cycloalkyl, C₃₋₈ cycloalkoxy, halo, hydroxy, nitro, oxo, optionally substituted C₁₋₁₀ heteroaryl, and optionally substituted phenyl.

More particularly “optionally substituted C₃₋₈ cycloalkyl” refers to a C₃₋₈ cycloalkyl optionally having from 1 to 3 substituents independently selected from the group consisting of C₁₋₄ alkyl, C₁₋₄ alkoxy, halo, and hydroxy.

The term “C₃₋₈ cycloalkoxy” refers to a C₃₋₈ cycloalkyl attached through an oxygen atom.

The terms “halogen” and “halo” refer to a chloro, fluoro, bromo or iodo atom.

The term “C₃₋₆ heterocycloalkyl” refers to a 4 to 10 membered monocyclic, saturated or partially (but not fully) unsaturated ring, having one to four heteroatoms selected from the group consisting of nitrogen, oxygen, and sulfur. It is understood that where sulfur is included that the sulfur may be —S—, —SO— or —SO₂—. The term includes, for example, azetidine, pyrrolidine, piperidine, piperazine, morpholine, thiomorpholine, oxetane, dioxolane, tetrahydropyran, tetrahydrothiopyran, tetrahydrofuran, hexahydropyrimidine, tetrahydropyrimidine, dihydroimidazole, and the like. It is understood that a C₃₋₆ heterocycloalkyl can be attached as a substituent through a ring carbon or a ring nitrogen atom.

More particularly, “C₃₋₆ heterocycloalkyl” is selected from the group consisting of pyrrolidine, piperidine, piperazine, morpholine, oxetane, tetrahydropyran, tetrahydrothiopyran, and tetrahydrofuran.

The term “optionally substituted C₃₋₆ heterocycloalkyl” refers to a C₃₋₆ heterocycloalkyl optionally substituted on the ring carbons with 1 to 4 substituents independently selected from the group consisting of optionally substituted C₁₋₄ alkyl, optionally substituted C₁₋₄ alkoxy, C₁₋₆ amide, C₁₋₇ amido, C₀₋₈ alkylamino, C₁₋₅ oxycarbonyl, cyano, optionally substituted C₃₋₈ cycloalkyl, C₃₋₈ cycloalkoxy, halo, hydroxy, nitro, oxo, and optionally substituted phenyl; and optionally substituted on any ring nitrogen with a substituent independently selected from the group consisting of optionally substituted C₁₋₄ alkyl, C₃₋₈ cycloalkyl, optionally substituted C₃₋₆ heterocycloalkyl, optionally substituted C₁₋₁₀ heteroaryl, and optionally substituted phenyl.

More particularly “optionally substituted C₃₋₆ heterocycloalkyl” refers to a C₃₋₆ heterocycloalkyl optionally substituted on the ring carbons with 1 to 4 substituents independently selected from the group consisting of C₁₋₄ alkyl, C₁₋₄ alkoxy, halo, and hydroxy and optionally substituted on any ring nitrogen with a C₁₋₄ alkyl.

The term “C₁₋₁₀ heteroaryl” refers to five to twelve membered monocyclic or polycyclic unsaturated, conjugated ring(s) having aromatic character and one to ten carbon atoms, and one or more, typically one to four, heteroatoms selected from the group consisting of nitrogen, oxygen, and sulfur. The term includes, for example, azepine, diazepine, furan, thiophene, pyrrole, imidazole, isothiazole, isoxazole, oxadiazole, oxazole, pyrazine, pyrazole, pyridazine, pyridine, pyrimidine, thiazole, thiadiazole, triazole, tetrazole, benzazepine, benzodiazepine, benzofuran, benzothiophene, benzimidazole, imidazopyridine, pyrazolopyridine, pyrrolopyridine, quinazoline, thienopyridine, indolizine, imidazopyridine, quinoline, isoquinoline, indole, isoindole, benzoxazole, benzoxadiazole, benzopyrazole, benzothiazole, and the like. It is understood that a C₁₋₁₀ heteroaryl can be attached as a substituent through a ring carbon or a ring nitrogen atom where such an attachment mode is available, for example for an indole, imidazole, azepine, triazole, pyrazine, etc.

More particularly, “C₁₋₁₀ heteroaryl” is selected from the group consisting of furan, thiophene, pyrrole, imidazole, isothiazole, isoxazole, oxadiazole, oxazole, pyrazine, pyrazole, pyridazine, pyridine, pyrimidine, thiazole, thiadiazole, and triazole.

The term “optionally substituted C₁₋₁₀ heteroaryl” refers to a C₁₋₁₀ heteroaryl optionally having 1 to 5 substituents on carbon independently selected from the group consisting of C₁₋₇ amido, C₀₋₈ alkylamino, C₁₋₆ amide, C₁₋₅ carbamoyl, C₁₋₆ sulfonylamido, C₀₋₆ sulfonylamino, C₁₋₅ ureido, optionally substituted C₁₋₄ alkyl, optionally substituted C₁₋₄ alkoxy, cyano, halo, hydroxy, oxo, nitro, C₁₋₅ oxycarbonyl, and C₁₋₈ sulfonyl, and optionally having a substituent on each nitrogen independently selected from the group consisting of optionally substituted C₁₋₄ alkyl, C₁₋₈ sulfonyl, optionally substituted C₃₋₆ heterocycloalkyl, and optionally substituted phenyl.

More particularly, “optionally substituted C₁₋₁₀ heteroaryl” refers to a C₁₋₁₀ heteroaryl optionally having 1 to 5 substituents on carbon independently selected from the group consisting of C₁₋₇ amido, C₀₋₈ alkylamino, C₁₋₆ amide, C₁₋₅ carbamoyl, C₁₋₆ sulfonylamido, C₀₋₆ sulfonylamino, C₁₋₅ ureido, C₁₋₄ alkyl, C₁₋₄ alkoxy, cyano, halo, hydroxy, oxo, C₁₋₅ oxycarbonyl, trifluoromethyl, trifluoromethoxy, and C₁₋₈ sulfonyl and optionally having a substituent on each nitrogen which is C₁₋₄ alkyl.

Even more particularly, “optionally substituted C₁₋₁₀ heteroaryl” refers to a C₁₋₁₀ heteroaryl optionally having 1 to 5 substituents independently selected from the group consisting of C₁₋₄ alkyl, C₁₋₄ alkoxy, cyano, halo, C₁₋₅ oxycarbonyl, trifluoromethyl, and trifluoromethoxy.

The term “oxo” refers to an oxygen atom having a double bond to the carbon to which it is attached to form the carbonyl of a ketone or aldehyde. It is understood that as the term is used herein oxo refers to doubly bonded oxygen attached to the group which has the oxo substituent, as opposed to the oxo group being pendant as a formyl group. For example, an acetyl radical is contemplated as an oxo substituted alkyl group and a pyridone radical is contemplated as an oxo substituted C₁₋₁₀ heteroaryl.

The term “C₁₋₁₀ heteroaryloxy” refers to a C₁₋₁₀ heteroaryl attached through an oxygen.

The term “optionally substituted C₁₋₁₀ heteroaryloxy” refers to a C₁₋₁₀ heteroaryl optionally having 1 to 5 substituents on carbon independently selected from the group consisting of C₁₋₄ alkyl, C₁₋₄ alkoxy, cyano, halo, hydroxy, nitro, oxo, C₁₋₈ sulfonyl, and trifluoromethyl and optionally having substituents on each nitrogen independently selected from the group consisting of optionally substituted C₁₋₄ alkyl, C₁₋₈ sulfonyl, and optionally substituted phenyl.

The term “optionally substituted phenyl” refers to a phenyl group optionally having 1 to 5 substituents independently selected from the group consisting of C₁₋₄ alkyl, C₁₋₄ alkoxy, C₁₋₉ amide, C₀₋₈ alkylamino, C₁₋₅ oxycarbonyl, cyano, halo, hydroxy, nitro, C₁₋₈ sulfonyl, and trifluoromethyl.

More particularly, “optionally substituted phenyl” refers to a phenyl group optionally having 1 to 5 substituents independently selected from the group consisting of C₁₋₄ alkyl, C₁₋₄ alkoxy, C₁₋₉ amide, C₀₋₈ alkylamino, C₁₋₅ oxycarbonyl, cyano, halo, hydroxy, nitro, and trifluoromethyl.

The term “C₁₋₆ sulfonylamido” refers to —NHS(O)₂R, wherein R is C₁₋₆ alkyl.

The term “C₀₋₆ sulfonylamino” refers to —S(O)₂NHR, wherein R is selected from the group consisting of hydrogen and C₁₋₆ alkyl.

The term “C₁₋₄ thioalkoxy” refers to a C₁₋₄ alkyl attached through a sulfur atom.

“Isomers” mean compounds having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are non-superimposable mirror images are termed “enantiomers” or sometimes “optical isomers.” A carbon atom bonded to four non-identical substituents is termed a “chiral center.” A compound with one chiral center has two enantiomeric forms of opposite chirality. A mixture of the two enantiomeric forms is termed a “racemic mixture.” A compound that has more than one chiral center has 2n-1 enantiomeric pairs, where n is the number of chiral centers. Compounds with more than one chiral center may exist as ether an individual diastereomer or as a mixture of diastereomers, termed a “diastereomeric mixture.” When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R and S sequencing rules of Cahn, Ingold and Prelog. For a given enantiomer, its “opposite enantiomer” is obtained by inverting the absolute configuration of each chiral center of the given enantiomer. Conventions for stereochemical nomenclature, methods for the determination of stereochemistry and the separation of stereoisomers are well known in the art. See, e.g., Michael B. Smith and Jerry March, Advanced Organic Chemistry (5th ed, 2001). In the chemical formulas depicted herein, one or more wedge bonds are used to designate absolute stereochemical configuration; the lack of a wedge bond at a chiral center indicates mixed or unspecified stereochemical configuration.

“Leaving group” means the group with the meaning conventionally associated with it in synthetic organic chemistry, i.e., an atom or group displaceable under reaction (e.g., alkylating) conditions. Examples of leaving groups include, but are not limited to, halo (e.g., F, Cl, Br and I), alkyl (e.g., methyl and ethyl) and sulfonyloxy (e.g., mesyloxy, ethanesulfonyloxy, benzenesulfonyloxy and tosyloxy), thiomethyl, thienyloxy, dihalophosphinoyloxy, tetrahalophosphoxy, benzyloxy, isopropyloxy, acyloxy, and the like.

Disclosed compounds may form pharmaceutically acceptable salts. These salts include acid addition salts (including di-acids) and base salts. Pharmaceutically acceptable acid addition salts include salts derived from inorganic acids such as hydrochloric acid, nitric acid, phosphoric acid, sulfuric acid, hydrobromic acid, hydroiodic acid, hydrofluoric acid, and phosphorous acids, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts include acetate, adipate, aspartate, benzoate, besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate, hydrogen phosphate, dihydrogen phosphate, pyroglutamate, saccharate, stearate, succinate, tannate, tartrate, tosylate, trifluoroacetate and xinofoate salts.

Pharmaceutically acceptable base salts include salts derived from bases, including metal cations, such as an alkali or alkaline earth metal cation, as well as amines. Examples of suitable metal cations include sodium, potassium, magnesium, calcium, zinc, and aluminum. Examples of suitable amines include arginine, N,N′-dibenzylethylenediamine, chloroprocaine, choline, diethylamine, diethanolamine, dicyclohexylamine, ethylenediamine, glycine, lysine, N-methylglucamine, olamine, 2-amino-2-hydroxymethyl-propane-1,3-diol, and procaine. For a discussion of useful acid addition and base salts, see S. M. Berge et al., J. Pharm. Sci. (1977) 66:1-19; see also Stahl and Wermuth, Handbook of Pharmaceutical Salts: Properties, Selection, and Use (2002).

Pharmaceutically acceptable salts may be prepared using various methods. For example, a compound may be reacted with an appropriate acid or base to give the desired salt. Alternatively, a precursor of the compound may be reacted with an acid or base to remove an acid- or base-labile protecting group or to open a lactone or lactam group of the precursor. Additionally, a salt of the compound may be converted to another salt through treatment with an appropriate acid or base or through contact with an ion exchange resin. Following reaction, the salt may be isolated by filtration if it precipitates from solution, or by evaporation to recover the salt. The degree of ionization of the salt may vary from completely ionized to almost non-ionized.

The term “substituted,” including when used in “optionally substituted” refers to one or more hydrogen radicals of a group having been replaced with non-hydrogen radicals (substituent(s)). It is understood that the substituents may be either the same or different at every substituted position and may include the formation of rings. Combinations of groups and substituents envisioned by this invention are those that are stable or chemically feasible.

The term “stable” refers to compounds that are not substantially altered when subjected to conditions to allow for their production. In a non-limiting example, a stable compound or chemically feasible compound is one that is not substantially altered when kept at a temperature of 40° C. or less, in the absence of moisture or other chemically reactive conditions, for about a week.

A disclosed compound is considered optically or enantiomerically pure (i.e., substantially the R-form or substantially the S-form) with respect to a chiral center when the compound is about 90% ee (enantiomeric excess) or greater; preferably equal to or greater than 95% ee; more preferably equal to or greater than 98% ee; and even more preferably equal to or greater than 99% ee with respect to a particular chiral center. A compound of the invention is considered to be in enantiomerically-enriched form when the compound has an enantiomeric excess of greater than about 1% ee; preferably greater than about 5% ee; and more preferably, greater than about 10% ee with respect to a particular chiral center.

It is understood that, where the terms defined herein mention a number of carbon atoms, that the mentioned number refers to the mentioned group and does not include any carbons that may be present in any optional substituent(s) thereon.

In addition, atoms making up the compounds of the present invention are intended to include all isotopic forms of such atoms. Isotopes, as used herein, include those atoms having the same atomic number but different mass numbers. For example, isotopes of hydrogen include tritium and deuterium, and isotopes of carbon include ¹³C and ¹⁴C.

The following abbreviations are used throughout the specification: Ac (acetyl); ACN (acetonitrile); Boc (tert-butoxycarbonyl); DBU (1,8-diazabicyclo[5.4.0]undec-7-ene); DCC (1,3-dicyclohexylcarbodiimide); DCM (dichloromethane); DMA (N,N-dimethylacetamide); DMAP (4-dimethylaminopyridine); DMF (N,N-dimethylformamide); DMSO (dimethylsulfoxide); EDCI (N-(3 -dimethylaminopropyl)-N′-ethylcarbodiimide); ee (enantiomeric excess); equiv (equivalents); Et (ethyl); EtOAc (ethyl acetate); EtOH (ethanol); HOBt (1H-benzo [d][1,2,3]triazol-1-ol); IPA (isopropanol); IPAc (isopropyl acetate); LDA (lithium diisopropylamide); LiHMDS (lithium bis(trimethylsilyl)amide); Me (methyl); MEK (methyl ethyl ketone); MeOH (methanol); MTBE (methyl tert-butyl ether); NaOt-Bu (sodium tertiary butoxide); NMM (N-methylmorpholine); NMP (N-methyl-2-pyrrolidinone); Ph (phenyl); Pr (propyl); i-Pr (isopropyl); RT (room temperature, approximately 20° C. to 25° C.); THF (tetrahydrofuran); TMS (trimethylsilyl); and Ts (tosyl).

### DETAILED DESCRIPTION OF THE INVENTION

Compounds produced according to the present invention may be synthesized according to the reaction schemes shown below. It should also be appreciated that a variety of different solvents, temperatures and other reaction conditions can be varied to optimize the yields of the reactions.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. Wuts, Protecting Groups in Organic Chemistry (1999) and P. Kocienski, Protective Groups (2000).

Certain compounds according to the present invention have atoms with linkages to other atoms that confer a particular stereochemistry to the compound (e.g., chiral centers). It is recognized that synthesis of compounds according to the present invention may result in the creation of mixtures of different stereoisomers (i.e., enantiomers and diastereomers). Unless a particular stereochemistry is specified, recitation of a compound is intended to encompass all of the different possible stereoisomers.

As used herein the symbols and conventions used in these processes, schemes and examples are consistent with those used in the contemporary scientific literature, for example, the Journal of the American Chemical Society or the Journal of Biological Chemistry. Unless otherwise noted, all starting materials were obtained from commercial suppliers and used without further purification.

All references to ether or Et₂O are to diethyl ether; and brine refers to a saturated aqueous solution of NaCl. Unless otherwise indicated, all temperatures are expressed in ° C. (degrees Centigrade). All reactions are conducted under an inert atmosphere at room temperature (RT) unless otherwise noted.

In each of the following reaction procedures or schemes, all substituents, unless otherwise indicated, are as previously defined.

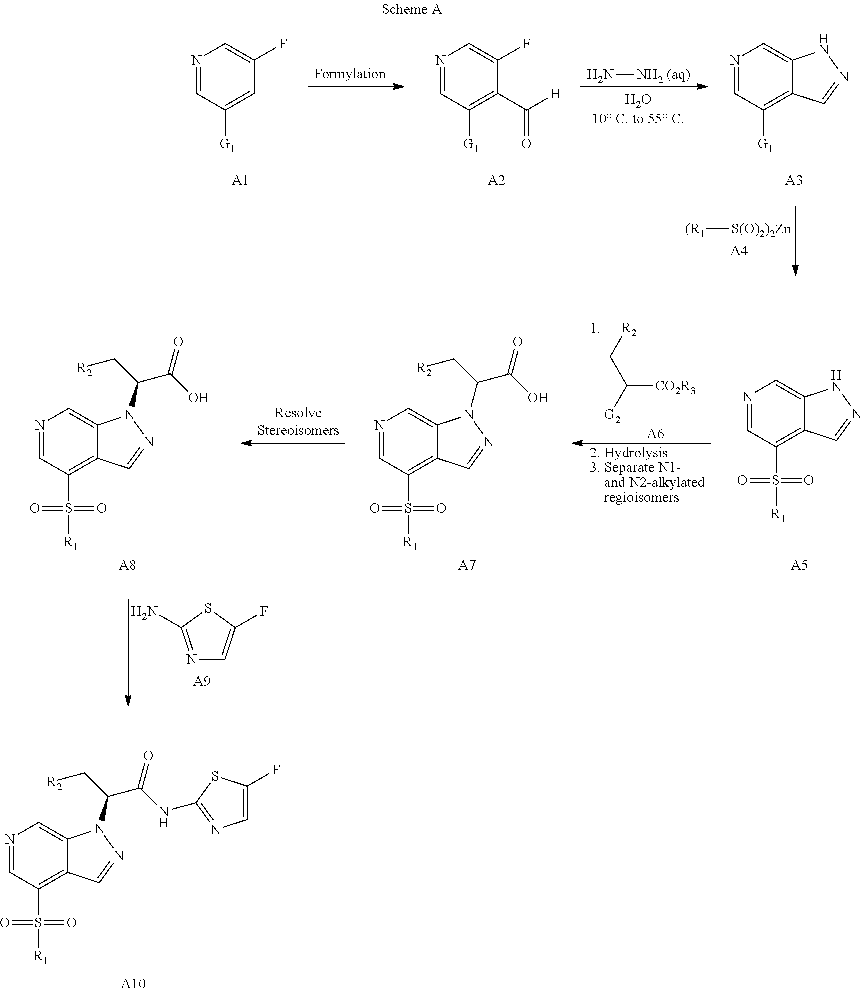

Scheme A shows a method for making azaindazole derivatives A10. In accordance with the method, an appropriately-substituted pyridine Al is formylated via treatment with a strong non-nucleophilic base (e.g., an amide base such as LDA, LiHMDS, NaHMDS, KHMDS, etc.) and reaction with an electrophile (e.g., methyl formate, DMF, etc.) in a suitable solvent (e.g., THF) at reduced temperature (e.g., <−70° C. for LDA or about −30° C. for LiHMDS), where G₁ in formula Al1 is a leaving group (e.g., halo, such as fluoro). Treatment of the resulting 3-fluoro-4-formylpyridine A2 with aqueous hydrazine at a temperature of about 10° C. to about 55° C. gives a hydrazone (e.g., a 3-fluoro-4-(hydrazonomethyl)pyridine, not shown) which cyclizes upon heating. The resulting indazole A3 is reacted with zinc (II) sulfinate A4, typically in an aqueous solution and at elevated temperature (up to 100° C.), to form R₁(indazol-4-yl)sulfone A5, which is subsequently reacted with a halo ester A6 in the presence of a base (e.g., inorganic base such as Cs₂CO₃, LiOt-Bu, Li₂CO₃, CsHCO₃, CsOH.H₂O, etc.), where G₂ in formula A6 is a leaving group (e.g., halo, such as bromo). The alkylation is generally carried out at a temperature of from about 0° C. to about 55° C. in an inert solvent (e.g., MEK, DMF, DMSO, THF, NMP, DMA, IPA, EtOAc, ACN, and the like) and gives, following hydrolysis, an N1-alkylated indazole A7 and an N2-alkylated regioisomer (not shown). Racemic N1-alkylated indazole A7 is isolated via, for example, trituration with isopropanol, and resolved to give a desired enantiomer A8.

Racemate A7 may be resolved through treatment with a chiral amine, subsequent separation of the diastereomeric salts, and regeneration of the chiral free acid A8. The opposite enantiomer (not shown) may be recovered, racemized, and recycled. For example, racemic acid A7 may be treated with chiral amine, (R)-N-(4-(dimethylamino)benzyl)-1-phenylethanaminium, to form a diastereomeric salt that may be crystallized from a variety of solvent systems, including H₂O, IPA, IPAc, MeOH, EtOH, and mixtures thereof. Useful solvent systems include binary mixtures of IPA and H₂O (7.8:0.5 v/v); IPAc and MeOH (20:2); IPAc and MeOH (15:1.5); and IPAc and EtOH (20:2), which may provide enantiomer A8 in enantiomeric excess (ee) of 95% or greater. For a detailed description of techniques that can be used to resolve stereoisomers, see Jean Jacques Andre Collet & Samuel H. Wilen, Enantiomers, Racemates and Resolutions (1981).

As shown in Scheme A, the chiral acid A8 is reacted with 5-fluoro-thiazol-2-ylamine A9 to form desired azaindazole A10. The amidation is typically carried out in the presence of an amide coupling agent (e.g., EDCI, DCC, etc.), optional catalyst (HOBt, DMAP, etc.) and one or more solvents (e.g., ACN, DMF, DMSO, THF, DCM, etc.) at temperature that may range from about room temperature to about 45° C.

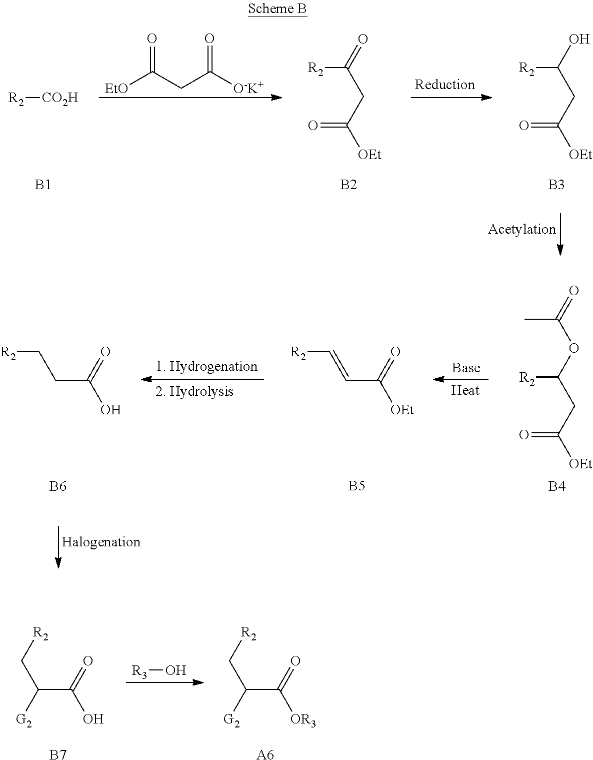

Scheme B shows a method for making halo esters A6. In accordance with the method, a β-keto ester B2, which is prepared from carboxylic acid B1 and ethyl malonate potassium salt, is reacted with a reducing agent (e.g., NaBH₄) to give β-hydroxy ester B3. Intermediate B3 is acetylated with, for example, acetic anhydride to form B4, which upon treatment with a non-nucleophilic base (e.g., DBU) at elevated temperature (e.g., about 50° C.) gives unsaturated ester B5. Hydrogenation of B5 gives a saturated ester (not shown) which is subsequently hydrolyzed via treatment with, for example, aqueous NaOH, to give an acid B6. Halogenation of the α-carbon atom (relative to the carboxy group) gives halo acid B7, which is reacted with R₃—OH, typically in the presence of a catalytic acid initiator (e.g., SOBr₂, TMSBr, HCl, H₂SO₄, p-TsOH, AcCl, and the like) to yield the desired ester A6. The α-halogenation may be carried out via conversion of B7 to a corresponding acid halide (e.g., acid chloride, not shown) followed by reaction with a halogen source (e.g., Br₂), aqueous work-up, and isolation of the halo acid A7. Alternatively, the halogenation and esterification steps shown in Scheme B may be carried out in a single pot, in which, following halogenation, the reaction is quenched with R₃—OH (e.g., methanol, ethanol, propanol, isopropanol, tert-butyl, etc.).

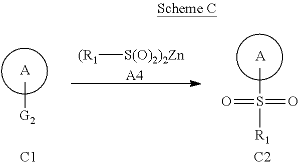

Scheme C shows a general method for preparing various sulfones C2. In accordance with the method, compound C1, which has a leaving group G₂ (e.g., halo, such as fluoro), is reacted with zinc (II) sulfinate A4 to form sulfone C2. The reaction is typically carried out in water, under neutral or slightly acidic conditions (e.g., in the presence of a weak acid such as KH₂PO₄), and at elevated temperature (up to 100° C.). The zinc (II) sulfinate A4 generally exists as a salt and may be represented by the following resonance structures:

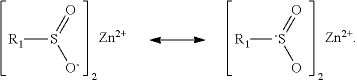

As noted earlier, compounds and intermediates shown in the schemes have substituent identifiers (A, R₁, R₂, R₃, G₁, and G₂) which are as defined above. Particular embodiments of the compounds and intermediates include those in which each of R₁ and R₂ is independently an optionally substituted C₁₋₆ alkyl, including methyl, ethyl, propyl or butyl; or is independently an optionally substituted C₃₋₈ cycloalkyl, including cyclopropyl, cyclobutyl, cyclopentyl or cyclohexyl; or is independently an optionally substituted C₃₋₆ heterocycloalkyl, including pyrrolidinyl, piperidinyl, piperazinyl, tetrahydropyranyl or tetrahydrofuranyl; or is independently an optionally substituted C₆₋₁₄ aryl, including phenyl; or is independently an optionally substituted C₁₋₁₀ heteroaryl, including pyridinyl or pyrazinyl.

In addition or as an alternative to the embodiments in the preceding paragraph, other embodiments include those in which R₃ is an optionally substituted C₁₋₆ alkyl, including methyl, ethyl, n-propyl, isopropyl, n-butyl, isobutyl, s-butyl or tert-butyl; or is methyl or ethyl; or is ethyl.

In addition or as an alternative to the embodiments in the preceding paragraphs, other embodiments include those in which A is optionally substituted C₁₋₁₀ heteroaryl.

In addition or as an alternative to the embodiments in the preceding paragraphs, other embodiments include those in which one or more of the substituents A, R₁, R₂, and R₃ are unsubstituted.

In addition or as an alternative to the embodiments in the preceding paragraphs, other embodiments include those in which G₁ is fluoro.

In addition or as an alternative to the embodiments in the preceding paragraphs, other embodiments include those in which G₂ is bromo.

### EXAMPLES

The present invention is further exemplified, but not limited by, the following examples.

### Example 1

### 3,5-Difluoroisonicotinaldehyde

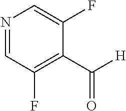

Anhydrous DMF (2.0 L) and anhydrous THF (5.0 L) were combined and the resulting mixture was cooled to −20° C. LiHMDS (10.4 L, 1.2 equiv) was added while maintaining the temperature between −15 and −25° C. The mixture was cooled to −30° C. and then 3,5-difluoropyridine (1.0 kg, 8.69 mol) was added while maintaining the temperature between −20° and −25° C. After one hour, the reaction mixture was added to a mixture of brine (4.0 kg NaCl in 16 L of DI water), THF (10 L), and concentrated aqueous HCl (2.2 L) at 0° C. The mixture was stirred for one hour and then the layers were separated. The pH of the aqueous layer was adjusted to about 7.5 with 2 N HCl solution (about 100 mL) and was extracted with MTBE/THF (1:1, 10 L). The organic layers were combined, washed with brine (1.0 kg NaCl in 4 L of DI water), and concentrated under reduced pressure to give the title compound as a yellow-orange, oily slurry.

### Example 2

### 4-Fluoro-1H-pyrazolo[3,4-c]pyridine

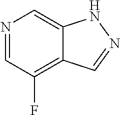

Crude 3,5-difluoroisonicotinaldehyde (2.0 kg) was suspended in DI water (6.0 L) and stirred to form a slurry. Hydrazine monohydrate (8.0 L) was cooled to a temperature of 10 to 15° C. The 3,5-difluoroisonicotinaldehyde/water slurry was slowly transferred to the hydrazine monohydrate to keep the internal temperature below 25° C. When the addition was complete, the mixture was gradually brought to 55° C. and was stirred at 55° C. for 40 hours and was then cooled to 0° C. and stirred for 18 hours before being filtered. The filter cake was washed with water (2×1.0 L) and was dried under vacuum (<3 in. Hg) at 35 to 40° C. for 24 hours to give a first crop of the title compound as an orange solid (884 g). The filtrate was extracted three times with 2-methyl THF (6.0 L). The organic layers were combined, washed with brine (4.0 L), and concentrated by rotary evaporation to give a residue which was slurried in a mixture of EtOAc/heptane (3:2, 4.0 L) for three hours. The slurry was filtered. The filter cake was washed with a mixture of EtOAc/heptane (3:2, 2×1.0 L) and dried under vacuum (<3 in. Hg) at 35-40° C. for 24 hours to give a second crop of the title compound (206 g).

### Example 3

### Zinc (II) cyclopropylsulfinate

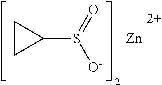

Zinc dust (<10 micron, 2.05 kg, 1.1 equiv) was slurried in EtOH (32 L) with agitation and then heated to a temperature of 70 to 75° C. Cyclopropanesulfonyl chloride (4.0 kg, 28.4 mol) was added while maintaining the internal temperature of the batch between 70 and 75° C. The mixture was then stirred for about one hour at 70° C., forming an off-white fine slurry. The mixture was filtered at 60 to 70° C. through a pad of Celite®, which was washed with EtOH (2×4 L). After 30 minutes, the filtrate was cooled to a temperature of 20 to 25° C. with agitation and then water (2 L) was slowly added over 30 to 45 minutes, forming a white slurry. The slurry was stirred for 18 hours at 20 to 25° C., cooled to a temperature of 0 to 5° C., and stirred for one hour before being filtered. The filter cake was washed with EtOH (2×4 L) and then dried under vacuum (<3 in. Hg) at 35 to 40° C. for 48 hours to give the title compound (4.037 kg). Karl Fisher analysis gave 12.03% water.

### Example 4

### 4-(Cyclopropylsulfonyl)-1H-pyrazolo[3,4-c]pyridine)

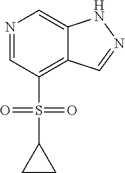 [SMILES: O=S(=O)(c1cncc2[nH]ncc12)C1CC1]

4-Fluoro-1H-pyrazolo[3,4-c]pyridine (1.50 kg, 10.9 mol), potassium phosphate monobasic (4.47 kg, 3.0 equiv), zinc (II) cyclopropyl sulfinate (3.07 kg, 0.9 equiv), and DI water (7.50 L) were combined and stirred, forming a thick brown slurry, which was subsequently heated to 100° C. After 45 hours, the mixture was cooled to 55° C. and EtOAc (15 L) was added. The mixture was stirred at 50 to 55° C. for two hours, cooled to a temperature of 20 to 25° C., and filtered over a pad of Celite®, which was rinsed with EtOAc (1.50 L). The layers were separated and the aqueous layer was extracted with EtOAc (6.0 L). The combined organic layers were washed with aqueous NaHCO₃ (5.0 wt %, 7.50 L), separated, and concentrated at 35 to 40° C. by rotary evaporation to give a slurry. Heptane (7.5 L) was added to the slurry, which was rotated on the rotary evaporator at 20 to 25° C. under atmospheric pressure for two hours. The slurry was filtered. The filter cake was washed with heptane (3.0 L) and dried under vacuum (<3 in. Hg) at 35 to 40° C. for 72 hours to give the title compound (1.922 kg; 90% purity by HPLC).

### Example 5

### Ethyl 3-oxo-3-(tetrahydro-2H-pyran-4-yl)propanoate

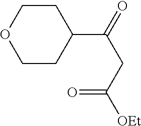 [SMILES: CCOC(=O)CC(=O)C1CCOCC1]

Ethyl malonate potassium salt (1.25 equiv, 1061 g) and THF (3.25 L) were combined in a first vessel and cooled to a temperature of 10 to 15° C. MgCl₂ (1.25 equiv, 594 g) was added slowly over 30 minutes, increasing the temperature to about 24° C. The mixture was heated at 50° C. for 2 hours and then cooled to 30° C. 1,1′-Carbonyldiimidazole (1.1 equiv, 891 g) and THF (1.62 L) were combined in a second vessel and tetrahydro-2H-pyran-4-carboxylic acid (1 equiv, 650 g) in THF (1.62 mL) was added over 30 minutes via an addition funnel, which was rinsed with THF (325 mL). After stirring 1.5 hours, this mixture in the second vessel was added to the first vessel over 30 minutes, increasing the temperature to about 34° C. The second vessel was rinsed with THF (325 mL) and the rinse solution was added to the reaction mixture (first vessel), which was heated at 30° C. for 16 hours. The reaction mixture was subsequently cooled to a temperature of 0 to 5° C., and aqueous HCl (3M, 6.5 L) was added over 30 minutes, causing the temperature to increase to about 25° C. The aqueous layer was separated from the THF layer, and was extracted with THF (2×5 volumes). The organic layers were combined and washed with a solution of Na₂CO₃ (20% in H₂O, 3.25 L), followed by brine (3.25 L). The organic layer was concentrated by rotary evaporation to give the title compound as a crude mixture.

### Example 6

### Ethyl 3-hydroxy-3-(tetrahydro-2H-pyran-4-yl)propanoate

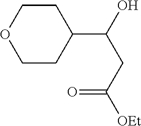 [SMILES: CCOC(=O)CC(O)C1CCOCC1]

The mixture from EXAMPLE 5 was cooled to a temperature of 10 to 15° C. and solid NaBH₄ (77 g, 0.4 equiv based on tetrahydro-2H-pyran-4-carboxylic acid) was added in portions over 25 minutes, increasing the temperature to about 39° C. Gas evolution was observed during the addition. The mixture was stirred at 20 to 25° C. for 1 hour, cooled to 0 to 5° C., treated with aqueous 2 N HCl (1.3 L), and diluted with isopropyl acetate (5 volumes). The layers were separated and the aqueous layer was extracted with of isopropyl acetate (5 volumes). The combined organic phases were washed with brine (3.25 L) and concentrated to approximately 1 volume of solvent. Isopropyl acetate (5 volumes) was added and removed by rotary evaporation to give the title compound (844 g).

### Example 7

### (Z)-Ethyl 3-(tetrahydro-2H-pyran-4-yl)acrylate

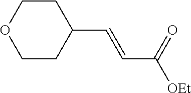 [SMILES: CCOC(=O)/C=C/C1CCOCC1]

To a mixture of ethyl 3-hydroxy-3-(tetrahydro-2H-pyran-4-yl)propanoate, THF (4.2 L), and DMAP (102 g, 0.2 equiv), was added acetic anhydride (435 mL, 1.1 equiv) at a rate to keep the internal temperature below 35° C. The mixture was stirred at room temperature for 3 hours. Next, DBU (750 mL, 1.2 equiv) was added to the mixture at a rate to keep the internal temperature below 35° C. The mixture was subsequently heated to 50° C. and stirred. After 16 hours, an additional 10% DBU was added, and the mixture was stirred for 8 more hours. The mixture was then cooled to a temperature of 20 to 25° C., diluted with MTBE (2.5 L), and extracted with aqueous 2 N HCl (4.2 L). The phases were separated, and the aqueous layer was extracted with MTBE (5 volumes). The combined organic layers were washed with brine (5 volumes) and then concentrated under reduced pressure to give an oil, which was dissolved in isopropyl acetate (3 L) and washed with 10% Na₂CO₃ (3 L). The organic layer was concentrated to give the title compound as a brown oil (716 g).

### Example 8

### 3-(Tetrahydro-2H-pyran-4-yl)propanoic acid

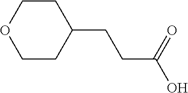 [SMILES: O=C(O)CCC1CCOCC1]

To a solution of (Z)-ethyl 3-(tetrahydro-2H-pyran-4-yl)acrylate (1 equiv, 716 g) dissolved in EtOH (2.8 L) was added PdOH₂ (3 wt %, 21.5 g) followed by the addition of hydrogen at a pressure of 3 psi (20 kpa), which caused an increase in temperature to about 30° C. over 1 hour. After 4 hours, the reaction was filtered over Celite® and washed with EtOH (720 mL). The filtrates from the hydrogenation were combined with 50% NaOH (2 equiv, 570 mL) and H₂O (720 mL) and stirred for 16 hours, after which the EtOH was largely removed by rotary evaporation. Water (2 volumes) was added and the resulting slurry was cooled to a temperature of 0 to 5° C. The pH of the slurry was adjusted from 14 to 1 with concentrated HCl (990 mL). The slurry was stirred for 1 hour and filtered. The filter cake was washed with water (1 volume), and dried under vacuum at 45° C. for 48 hours to give the title compound as a white solid (487 g).

### Example 9

### 2-Bromo-3-(tetrahydro-2H-pyran-4-yl)propanoic acid

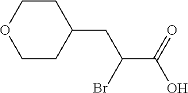 [SMILES: O=C(O)C(Br)CC1CCOCC1]

To a solution of 3-(tetrahydro-2H-pyran-4-yl)propanoic acid (1 equiv, 0.32 mol, 50.00 g) in chlorobenzene (250 mL) was added SOCl₂ (1.5 equiv, 0.47 mol, 34.5 mL) followed by DMF (5 mol %, 0.02 mol, 1.22 mL). The reaction mixture was stirred for 1.5 hours at 21° C. Bromine (1.5 equiv, 0.47 mol, 24.4 mL) was then added, and the reaction mixture was heated to 85 to 90° C. for 16 hours. Additional bromine (6.0 mL) was added and the reaction mixture was heated at the same temperature for 4 more hours. The reaction mixture was subsequently cooled in an ice bath to a temperature of 0 to 5° C. Water (10 equiv, 57 mL) was added via an addition funnel and the mixture was stirred for 21 hours. Water (15 mL) was then added to drive the reaction to completion. The resulting slurry was cooled and filtered. The filter cake was washed with chlorobenzene (50 mL) and dried under vacuum at 45° C. for 20 hours to give the title compound (41.53 g, 55% yield).

### Example 10

### Ethyl 2-bromo-3-(tetrahydro-2H-pyran-4-yl)propanoate

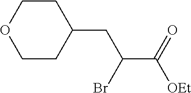 [SMILES: CCOC(=O)C(Br)CC1CCOCC1]

2-Bromo-3-(tetrahydro-2H-pyran-4-yl)propanoic acid (6.0 kg, 25.5 mol, 1.00 equiv) was suspended in EtOH (24.0 L). Thionyl bromide (1.98 L, 0.1 equiv) was slowly added via an addition funnel while maintaining an internal temperature below 40° C. The reaction mixture was heated to a temperature of 55 to 60° C., stirred for 16 hours, cooled to 20° C. and concentrated by rotary evaporation to give a residue. The residue was combined with EtOAc (12.0 L) and DI H₂O (6.0 L) and was agitated before the phases were allowed to separate. The organic layer was separated and the aqueous layer was extracted with EtOAc (12.0 L). The organic layers were combined, washed with a 20 wt % saturated aqueous brine solution (9.6 L) followed by DI water (2.4 L) and concentrated by rotary evaporation to give the title compound as an orange, viscous oil (6.907 kg, 96.6% yield; 94.5% pure by HPLC (AUC)).

### Example 11

### 2-(4-(Cyclopropylsulfonyl)-1H-pyrazolo[3,4-c]pyridin-1-yl)-3-(tetrahydro-2H-pyran-4-yl)propanoic acid

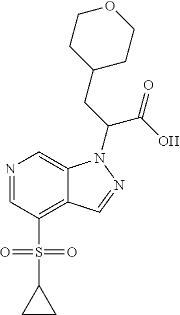 [SMILES: O=C(O)C(CC1CCOCC1)n1ncc2c(S(=O)(=O)C3CC3)cncc21]

To a mixture of 4-(cyclopropylsulfonyl)-1H-pyrazolo[3,4-c]pyridine (5.0 kg, 22.4 mol, 1.00 equiv) and MEK (5 volumes) was added Cs₂CO₃ (14.594 kg, 44.8 mol, 2.00 equiv) portion-wise over the course of about 17 minutes. A solution of ethyl 2-bromo-3-(tetrahydro-2H-pyran-4-yl)propanoate (6.410 kg, 22.8 mol, 1.02 equiv-based on 94.5 wt %) in MEK (4 volumes) was then added drop-wise over about 48 minutes. After 1 hour the reaction mixture was heated to 54° C. and stirred for 12 hours. The reaction mixture was cooled to 12° C. and NaOH (7.665 kg) was added over about 53 minutes. The reaction mixture was then stirred for 50 minutes at 18° C., after which DI H₂O (4 volumes) and isopropyl acetate (4 volumes) were added. The reaction mixture was agitated and the layers were allowed to separate. The aqueous layer was separated and the organic layer was back-extracted with aqueous 2 N NaOH (1 volume). The aqueous layers were combined and partitioned between isopropyl acetate/THF (4:1, 8 volumes). The pH of the biphasic solution was adjusted to 3.2 with aqueous 6 N HCl (5 volumes) over the course of 3 hours. An additional 500 g of concentrated HCl was added and the layers were allowed to separate. The aqueous phase was separated and back-extracted with isopropyl acetate/THF (4:1, 5 volumes). The organic layers were combined and washed with aqueous 1 N HCl/20 wt % brine solution (1:1). The organic layer was washed with a 16 wt % brine solution, separated, agitated overnight, and subsequently reduced to 4 volumes under reduced pressure. Isopropanol (4 volumes) was added and the total volume was again reduced to 4 volumes at reduced pressure. IPA (4 volumes) was again added and the total volume was again reduced to 4 volumes at reduced pressure before being cooled to 20° C. and filtered. The filter cake was washed with IPA (2×2 volumes) then dried under vacuum at 30° C. to a constant weight to give the title compound as a pale orange-taupe solid (3.725 kg).

### Example 12

### (S)-2-(4-(Cyclopropylsulfonyl)-1H-pyrazolo[3,4-c]pyridin-1-yl)-3-(tetrahydro-2H-pyran-4-yl)propanoate, (R)-N-(4-(dimethylamino)benzyl)-1-phenylethanaminium salt

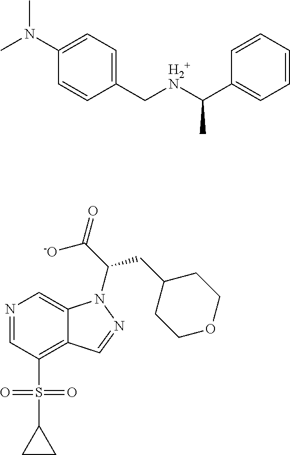

2-(4-(Cyclopropylsulfonyl)-1H-pyrazolo[3,4-c]pyridin-1-yl)-3-(tetrahydro-2H-pyran-4-yl)propanoic acid (514 g, 1.36 mol, 1.00 equiv) was combined with IPA (2.06 L) and heated to 70° C. (R)-N,N-Dimethyl-4-((1-phenylethylamino)methyl)aniline (345.4 g, 1.36 mol, 1.00 equiv) was added in IPA (0.775 L, 1.5 volumes) drop-wise over the course of 45 minutes, maintaining an internal temperature of 70° C. The addition funnel was rinsed with IPA (0.5 volumes). The mixture was agitated for 20 minutes, treated with of DI H₂O (21 mL, 0.01 equiv), then cooled to 55° C. gradually over the course of 45 minutes. The mixture was seeded with the enantiomerically-enriched title compound (2.42 g, 0.005 mass equiv), gradually cooled to ambient temperature over the course of 4 hours, and agitated overnight. The mixture was subsequently cooled to 0° C. and filtered. The filter cake was rinsed with IPA (2×1 volume), cooled to 0° C., dried under vacuum for 0.75 hours, and then placed in a vacuum oven at 30° C. overnight to give the title compound as a pale yellow solid (364.6 g).

(S)-2-(4-(Cyclopropylsulfonyl)-1H-pyrazolo [3,4-c]pyridin-1-yl)-3-(tetrahydro-2H-pyran-4-yl)propanoate, (R)-N-(4-(dimethylamino)benzyl)-1-phenylethanaminium salt (6.986 kg, 11.02 mol, 1.00 equiv) was combined with IPA (7.8 volumes) and DI H₂O (350 mL), heated to 75° C. and stirred for 1.5 hours. The reaction mixture was gradually cooled to 21° C. over 2 hours and subsequently cooled to 2° C., where it was held for 1 hour, then filtered. The vessel was rinsed with IPA (2×2 volumes). The filter cake was washed with the IPA rinses, conditioned overnight under reduced pressure and an atmosphere of nitrogen, and dried to a constant mass at 35° C. under reduced pressure to give the title compound (chiral purity of 97.8%).

### Example 13

### (S)-2-(4-(Cyclopropylsulfonyl)-1H-pyrazolo [3,4-c]pyridin-1-yl)-3-(tetrahydro-2H-pyran-4-yl)propanoic acid

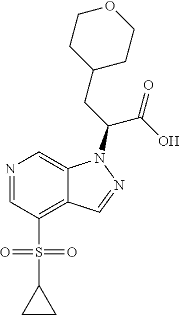 [SMILES: O=C(O)[C@H](CC1CCOCC1)n1ncc2c(S(=O)(=O)C3CC3)cncc21]

(S)-2-(4-(Cyclopropylsulfonyl)-1H-pyrazolo[3,4-c]pyridin-1-yl)-3-(tetrahydro-2H-pyran-4-yl)propanoate, (R)-N-(4-(dimethylamino)benzyl)-1-phenylethanaminium salt (6.178 kg, 9.75 mol, 1.00 equiv), IPA (6.2 L), and 1 N aqueous HCl (18.6 L) were combined while maintaining an internal temperature at less than 25° C. The mixture was heated to 30° C., agitated for 1 hour, cooled to ambient temperature over the course of 1 hour, agitated for 4 hours, cooled to 0° C., and held at to 0° C. for 12 hours. The resulting slurry was filtered. The filter cake was successively rinsed with aqueous 0.5 N HCl (2 volumes) and DI H₂O/IPA (10:1, 2 volumes) and then dried at 35° C. under vacuum overnight to a constant weight, giving the title compound as a light-tan granular solid (3.200 kg).

### Example 14

### 2-(tert-Butoxycarbonylamino)thiazole-5-carboxylic acid

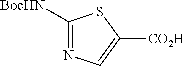 [SMILES: CC(C)(C)OC(=O)Nc1ncc(C(=O)O)s1]

A mixture of 2-aminothiazole-5-carboxylic acid (2.2 kg, 15.33 mol), aqueous 2 M NaOH (0.674 kg in 8.39 L of DI water), DI water (17.68 L), and THF (17.68 L) was cooled to about 0° C. A solution of Boc-anhydride (4.02 kg, 1.20 equiv) in THF (2.21 L) was added to the mixture while maintaining an internal temperature below 5° C. When the addition was complete, the reaction mixture was warmed to an internal temperature of 25° C. and was stirred for 24 hours. The reaction mixture was cooled to about 0° C. and diluted with DI water (22.1 L). While maintaining an internal temperature below 5° C., the pH of the mixture was adjusted to 4.9 by slowly adding acetic acid (5.30 L). After 1 hour a precipitate formed, which was collected by filtration, and rinsed successively with DI water (6.63 L) and MTBE (4.42 L). The filter cake was held under nitrogen for 1 hour and then dried under reduced pressure at 25° C. to afford the title compound (5.14 kg).

### Example 15

### tert-Butyl 5-fluorothiazol-2-ylcarbamate

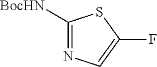 [SMILES: CC(C)(C)OC(=O)Nc1ncc(F)s1]

2-(tert-Butoxycarbonylamino)thiazole-5-carboxylic acid (2.06 kg, 8.43 mol) and 2-methyl THF (16.5 L) were combined and cooled to −5° C. Selectfluor® (5.975 kg, 2.0 equiv) was added in portions while maintaining an internal temperature below 5° C. Next, a solution of potassium phosphate (5.192 kg, 2.90 equiv) in DI water (16.5 L), which was cooled to a temperature of 0 to 5° C., was slowly added to the mixture while maintaining an internal temperature below 5° C. When the addition of the potassium phosphate solution was complete, the reaction mixture was filtered through a pad of Celite®, which was rinsed with 2-methyl THF (6.18 L). The organic and aqueous phases of the filtrate were separated. The aqueous layer was extracted with 2-methyl THF (2×6.18 L), and the organic layers were combined and washed successively with aqueous sodium bicarbonate (0.964 kg in 12.36 L DI water) (2×6.0 L), aqueous HCl (0.516 L), and brine (1.607 kg in 4.57 L DI water). The organic phase was concentrated to dryness at 45° C. and then dried under vacuum at 25° C. for approximately 2 days to give the title compound (3.756 kg).

### Example 16

### 5-Fluoro-thiazol-2-ylamine

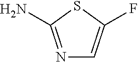

To a mixture of tert-butyl 5-fluorothiazol-2-ylcarbamate and 1,4-dioxane (13.34 L) was added anhydrous HCl gas (3.0 kg) over 5 hours via subsurface sparging. The mixture was purged with nitrogen for 1 hour. Next, MTBE (5.34 L) was slowly added and the mixture was cooled to a temperature between 0 and 5° C. After 1 hour, the solids were collected by filtration and rinsed with MTBE (2×5.34 L). The filter cake was held under nitrogen for 1 hour and then dried under vacuum at 25° C. to afford a tan solid. The crude product was slurried in water/THF (1.21 L:12.11 L) with agitation for 1 hour at ambient temperature. The solid was collected by filtration, rinsed with THF (2×5.3 L), and then dried under vacuum at 25° C. to afford an HCl salt of the title compound as an off-white solid.

### Example 17

### (S)-2-(4-(Cyclopropylsulfonyl)-1H-pyrazolo[3,4-c]pyridin-1-yl)-N-(5-fluorothiazol-2-yl)-3-(tetrahydro-2H-pyran-4-yl)propanamide

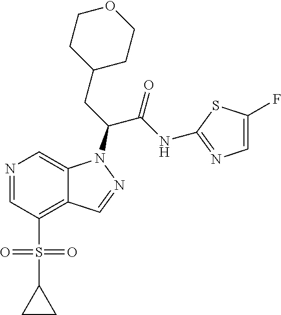 [SMILES: O=C(Nc1ncc(F)s1)[C@H](CC1CCOCC1)n1ncc2c(S(=O)(=O)C3CC3)cncc21]

(S)-2-(4-(Cyclopropylsulfonyl)-1H-pyrazolo[3,4-c]pyridin-1-yl)-3-(tetrahydro-2H-pyran-4-yl)propanoic acid (3.22 kg, 6.98 mol, 1.00 equiv), ACN (13.3 L), and an HCl salt of 5-fluoro-thiazol-2-ylamine (1.60 kg, 1.00 equiv, 0.5% water) were combined at ambient temperature. EDCI (2.68 kg, 2.00 equiv) was added in portions while maintaining an internal temperature below 30° C. The mixture was heated to 45° C. with continued agitation for 4 hours and then filtered. The pH of the filtrates was adjusted to 5.45 with sodium biphosphate (0.90 kg, 0.34 equiv in 17.0 L of DI water). After stirring at ambient temperature for 30 minutes, DI water (45.0 L) was added over a period of about 1 hour to give a slurry. The solids were collected by filtration, rinsed with DI water (5×7.95 L), evacuated under a rubber dam for 3 hour, then dried under vacuum at 35° C. for 72 hours to afford the title compound as a tan solid (2.86 kg).

1. A method of making a compound of formula 1, 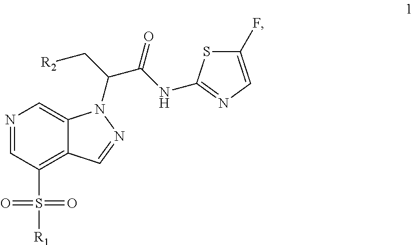
or a pharmaceutically acceptable salt thereof, the method comprising:
reacting a compound of formula A3 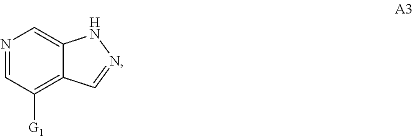
with a compound of formula A4,
(R₁—S(O)₂)₂Zn, A4
to give a compound of formula A5, 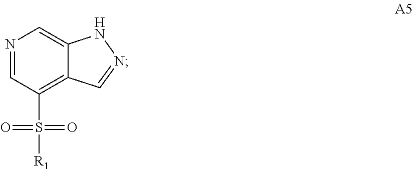
reacting the compound of formula A5 with a compound of formula A6, 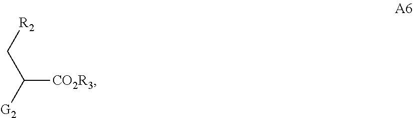
to give, following hydrolysis, a compound of formula A7, 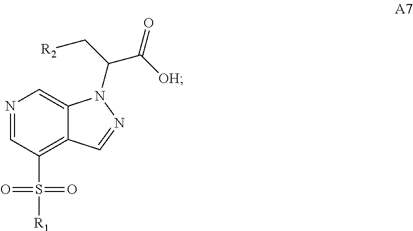
reacting the compound of formula A7 with a compound of formula A9, 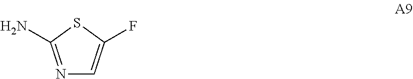
or a salt thereof, to give the compound of formula 1; and
optionally converting the compound of formula 1 to a pharmaceutically acceptable salt; wherein
G₁ and G₂ are each independently halo;
R₁ is selected from the group consisting of C₁₋₆ alkyl, C₃₋₈ cycloalkyl-C₁₋₆ alkyl, C₃₋₆ heterocycloalkyl-C₁₋₅ alkyl, C₆₋₁₄ aryl-C₁₋₆ alkyl, C₁₋₁₀ heteroaryl-C₁₋₆ alkyl, C₃₋₈ cycloalkyl, C₃₋₆ heterocycloalkyl, C₆₋₁₂ aryl, and C₁₋₁₀ heteroaryl, each optionally substituted;
R₂ is selected from the group consisting of hydrogen, halo, cyano, thio, hydroxy, C₁₋₅ carbonyloxy, C₁₋₄ alkoxy, C₆₋₁₄ aryloxy, C₁₋₁₀ heteroaryloxy, C₁₋₅ oxycarbonyl, C₁₋₉ amide, C₁₋₇ amido, C₀₋₈ alkylamino, C₁₋₆ sulfonylamido, imino, C₁₋₈ sulfonyl, C₁₋₆ alkyl, C₃₋₈ cycloalkyl-C₁₋₆ alkyl, C₃₋₆ heterocycloalkyl-C₁₋₆ alkyl, C₆₋₁₄ aryl-C₁₋₆ alkyl, C₁₋₁₀ heteroaryl-C₁₋₅ alkyl, C₃₋₈ cycloalkyl, C₃₋₆ heterocycloalkyl, C₆₋₁₄ aryl, and C₁₋₁₀ heteroaryl, each optionally substituted; and
R₃ is selected from the group consisting of (C₁₋₆)alkyl, (C₃₋₈)cycloalkyl, (C₃₋₆)hetero cyclo alkyl, (C₆₋₁₄)aryl, (C₁₋₁₀)hetero aryl, (C₃₋₈)cycloalkyl(C₁₋₆)alkyl, (C₃₋₆)heterocycloalkyl(C₁₋₆)alkyl, (C₆₋₁₄)aryl(C₁₋₆)alkyl, and (C₁₋₁₀)heteroaryl(C₁₋₆)alkyl, each optionally substituted.

2. The method according to claim 1, further comprising:
prior to reaction with the compound of formula A9, resolving the compound of formula A7 to obtain a compound of formula A8, 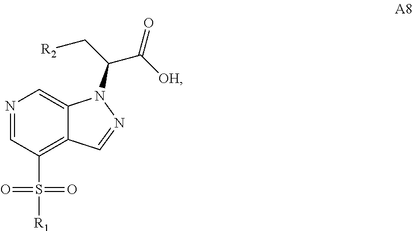
or an opposite enantiomer thereof, so as to form a compound of formula A10, 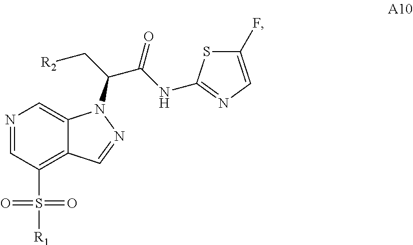
or an opposite enantiomer thereof.

3. The method according to claim 1, further comprising:
halogenating a compound of formula B6, 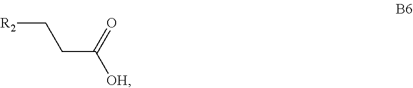
to give a compound of formula B7, 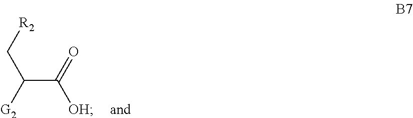
reacting the compound of formula B7 with R₃—OH to give the compound of formula A6.

4-14. (canceled)

15. The method according to claim 1, wherein R₁ and R₂ are each independently selected from the group consisting of C₁₋₆ alkyl, C₃₋₈ cycloalkyl, pyrrolidinyl, piperidinyl, piperazinyl, tetrahydropyranyl, tetrahydrofuranyl, phenyl, pyridinyl, and pyrazinyl, each optionally substituted.

16. The method according to claim 15, wherein R₁ is cyclopropyl.

17. The method according to claim 15, wherein R₂ is tetrahydro-2H-pyran-4-yl.

18. The method according to claim 1, wherein R₃ is C₁₋₆ alkyl.

19. The method according to claim 1, wherein R₃ is ethyl.

20. The method according to claim 1, wherein G₁ is fluoro.

21. The method according to claim 1, wherein G₂ is bromo.

22. A method of making a compound of formula A5, 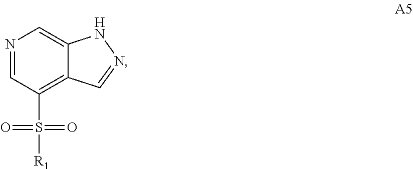
the method comprising:
reacting a compound of formula A3, 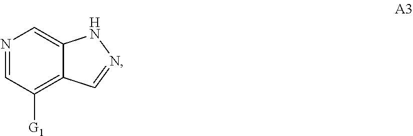
with a compound of formula A4,
(R₁—S(O)₂)₂Zn; A4
wherein
G₁ is halo; and
R₁ is selected from the group consisting of C₁₋₆ alkyl, C₃₋₈ cycloalkyl-C₁₋₆ alkyl, C₃₋₆ heterocycloalkyl-C₁₋₅ alkyl, C₆₋₁₄ aryl-C₁₋₆ alkyl, C₁₋₁₀ heteroaryl-C₁₋₆ alkyl, C₃₋₈ cycloalkyl, C₃₋₆ heterocycloalkyl, C₆₋₁₂ aryl, and C₁₋₁₀ heteroaryl, each optionally substituted.

23. The method according to claim 22, wherein R₁ is selected from the group consisting of C₁₋₆ alkyl, C₃₋₈ cycloalkyl, pyrrolidinyl, piperidinyl, piperazinyl, tetrahydropyranyl, tetrahydrofuranyl, phenyl, pyridinyl, and pyrazinyl, each optionally substituted.

24. The method according to claim 22, wherein R₁ is cyclopropyl.

25. The method according to claim 22, wherein G₁ is fluoro.

26. A method of making a compound of formula A6, 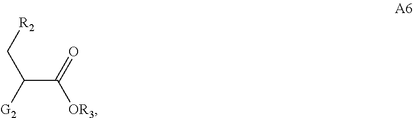
the method comprising:
halogenating a compound of formula B6, 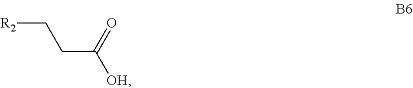
to give a compound of formula B7, 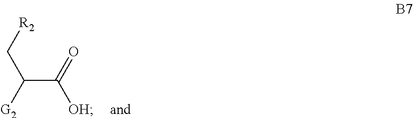
reacting the compound of formula B7 with R₃—OH;
wherein
G₂ is halo;
R₂ is selected from the group consisting of hydrogen, halo, cyano, thio, hydroxy, C₁₋₅ carbonyloxy, C₁₋₄ alkoxy, C₆₋₁₄ aryloxy, C₁₋₁₀ heteroaryloxy, C₁₋₅ oxycarbonyl, C₁₋₉ amide, C₁₋₇ amido, C₀₋₈ alkylamino, C₁₋₆ sulfonylamido, imino, C₁₋₈ sulfonyl, C₁₋₆ alkyl, C₃₋₈ cycloalkyl-C₁₋₆ alkyl, C₃₋₆ heterocycloalkyl-C₁₋₆ alkyl, C₆₋₁₄ aryl-C₁₋₆ alkyl, C₁₋₁₀ heteroaryl-C₁₋₅ alkyl, C₃₋₈ cycloalkyl, C₃₋₆ heterocycloalkyl, C₆₋₁₄ aryl, and C₁₋₁₀ heteroaryl, each optionally substituted; and
R₃ is selected from the group consisting of (C₁₋₆)alkyl, (C₃₋₈)cycloalkyl, (C₃₋₆)hetero cyclo alkyl, (C₆₋₁₄)aryl, (C₁₋₁₀)hetero aryl, (C₃₋₈)cycloalkyl(C₁₋₆)alkyl, (C₃₋₆)heterocycloalkyl(C₁₋₆)alkyl, (C₆₋₁₄)aryl(C₁₋₆)alkyl, and (C₁₋₁₀)heteroaryl(C₁₋₆)alkyl, each optionally substituted.

27. The method according to claim 26, wherein R₂ is selected from the group consisting of C₁₋₆ alkyl, C₃₋₈ cycloalkyl, pyrrolidinyl, piperidinyl, piperazinyl, tetrahydropyranyl, tetrahydrofuranyl, phenyl, pyridinyl, and pyrazinyl, each optionally substituted.

28. The method according to claim 26, wherein R₂ is tetrahydro-2H-pyran-4-yl.

29. The method according to claim 26, wherein R₃ is C₁₋₆ alkyl.

30. The method according to claim 26, wherein R₃ is ethyl.

31. The method according to claim 26, wherein G₂ is bromo.
